(19) United States
(12) Patent Application Publication
(10) Pub. No.: US 2010/0236709 A1
(43) Pub. Date: Sep. 23, 2010

(54) NOVEL SCENTS AND METHODS TO USE THE SAME

(76) Inventor: Donna Marie Mutch, Hicksville, NY (US)

Correspondence Address: Limin Wen, One Bungtown Road, Nichols Building, 1st Floor, Cold Spring Harbor, NY, 11724, US

(21) Appl. No.: 12/726,233

(22) Filed: Mar. 17, 2010

Related U.S. Application Data
(60) Provisional application No. 61/161,807, filed on Mar. 20, 2009.
(60) Provisional application No. 61/231,491, filed on Aug. 5, 2009.

(51) Int. Cl. A61K 8/97; B05D 1/02; B05D 1/28; B05D 1/18; A61Q 13/00; B32B 37/12; B23P 11/00; A61L 9/04

(52) U.S. Cl. 156/278; 427/421.1; 427/429; 427/430.1; 512/5; 29/525.01; 239/6

(57) ABSTRACT

Novel formulated scents and methods to deliver such scents with or without scent strips and reed diffuser through natural airflow by directly applying formulated scents to the screen or by directly attaching such scent strips to anywhere people want or with the use of reed diffusers, without any scent delivery device.

### REFERENCE TO RELATED APPLICATIONS

This application claims the benefit of the filing date of U.S. Provisional Application Ser. No. 61/161,807, entitled “SCREEN SCENTS” and filed on Mar. 20, 2009, and the benefit of the filing date of U.S. Provisional Application Ser. No. 61/231,491, entitled “SCENT STRIPS” and filed on Aug. 5, 2009. The teachings of the entire referenced applications are incorporated herein by references.

### FIELD OF THE INVENTION

The present invention relates to scents and, more particularly, regarding to formulated scents/scents which contain naturally occurring insect repellent, which can be applied to a screen or mesh and specially designed scent strips and reed diffusers. For example, such formulated scents could be applied to a door screen and/or a window screen, wherein air flows through the screen for dispersal of the scent. Also, some special designed scent strips could be infused with such formulated or natural fragrance(s) which can be attached to various situations or locations such as fans/air conditioners, a door screen, a window screen, outdoor structures including gazebos/tents, outdoor chairs, or the likes wherein fragrance scent can be diffused from the scent strips and reed diffusers by both natural and artificial airflow throughout the selected area.

### BACKGROUND OF THE INVENTION

Scent delivery devices are known that release scent from a reservoir when exposed to air. However, these devices are passive, such that the released scent remains concentrated in relatively close proximity to the devices.

Scent delivery devices are also known that are adapted for attachment to the outlets, or vents, of heating or cooling apparatuses. The artificial airflow generated by the apparatuses disperses the released scent. However, in certain climates and at certain times of the year it is preferable to open doors and windows. As heating and cooling apparatuses are normally turned off when doors and windows are open, the generated artificial airflow ceases and the scent is again concentrated in relatively close proximity to the devices.

All the known scent delivery devices have to be attached or fixed to the apparatuses or screen, they may block the view through windows or doors and they may damage the screen with which they attached. Also, making or buying such devices is an extra cost for people, plus most of them need to be plugged into an outlet.

It would be highly desirable if there were a simple and easy way to directly apply scents or attach scent dispenser without plugging into any outlet, or using either aerosol sprays or candles, which would allow artificial airflow generated by apparatuses to disperse the released scent. The apparatuses mentioned here include vents, fans, heating or cooling apparatuses such as air conditioners. Therefore, it is preferred to have novel fragrance scents containing naturally occurring insect repellent that can flow along with the airflow of the fans or air conditioners indoors and can be used in certain climates and at certain times of the year when people stay outdoors or open doors and windows.

Due to these and other problems and disadvantages encountered in the prior art, there is a need for a novel scent containing a naturally occurring insect repellent which could be applied directly to a screen or be attached through some kind of scent tags, strips or disseminated by reed diffusers at flexible locations.

### SUMMARY OF THE INVENTION

The present invention overcomes the above described and other problems and disadvantages by creating novel formulated scents, which was directly applied to a door or window screen or through special designed scent strips or reed diffusers exposed to a natural airflow for dispersing the scent over time.

In one embodiment, the present invention is to apply scents/scents that contain naturally occurring insect repellent to various screens that will include but not limited to door screens, window screens, tent screens and gazebos. This will allow the fragrances to be delivered by natural elements e.g. breezes.

There are various ways to apply formulated scents directly to related screens including but not limited to towelettes, fine spray misters, foam applicators, etc.

The ingredients in such formulated scents described here including, but not limited to, Mineral Oil, Isopropyl Palmitate, Dicapryl Adipate, Fragrance (such as orange-ginger combination), Dioctyl Sodium Sulfosuccinate, Butylated hydroxytoluene (BHT), Carrot Seed Oil.

In another embodiment, the present invention overcomes the above described and other problems and disadvantages by attaching scent strips directly to different location as desired, including but not limited to, fans/air conditioners, a door screen, a window screen, outdoor structures such as gazebos/tents, outdoor umbrellas, outdoor chairs, or the likes wherein fragrance scent can be diffused from the scent strips by natural airflow throughout the selected area.

The present invention is to attach different sizes of strips infused with formulated scents or natural fragrance to various locations as desired including indoors and outdoors. For example, such scent strips can be attached, but not limited to, to outside frames of fans/air conditioners within the house or buildings. Also, such scent strips can be hung in outdoor structures or furniture including gazebos/tents, umbrellas or swing chairs.

The scent strip invented was infused or soaked with formulated scents mentioned in the first embodiment with natural or artificial scents that also contain a naturally occurring insect repellant.

The said scent strips can be made from any suitable material including but not limited to paper, cloth, yarn, plastic, rubber, wood, stone, metal, etc. The size of the scent strip can be different depending on the situation or location. The shape of the scent strip can be any decorated shape including but not limited to rectangle, triangle, diamond, circle, square, or any suitable shape as desired. Additionally, the scent strip can be made like an animal, flower, or any natural or artificial shape. Moreover, the color of the scent strip can be made in any desirable color such as red, green, yellow, purple, blue, pink, or different combinations of colors.

Such an elegant strip is inexpensive to manufacture, easy to store in a sealed container for future use, and flexible to attach to any location as desired.

The mechanism to attach such scent strips can be any conventional means for such attachment including but not limited to using clips, hooks, adhesives and Velcro/elastic ways.

In another embodiment, the present invention overcomes the above described and other problems and disadvantages by using reed diffusers that are soaked or infused with formulated scents/scents containing naturally occurring insect repellent. Said reed diffusers can be placed on, but not limited to, outdoor dining/picnic tables and all other suitable outdoor surfaces as well as suitable indoor surfaces such as, but not limited to, end tables, dining and coffee tables, shelves etc.

The reed diffuser invented was infused or soaked with formulated scents mentioned in the first embodiment with natural or artificial scents that also contain a naturally occurring insect repellant.

The said reed diffusers can be made from any suitable material including but not limited to paper, cloth, yarn, plastic, rubber, wood, stone, metal, etc. The size of the reed diffuser can be different depending on the situation or location. The shape of the reed diffuser can be any decorated shape including but not limited to rectangle, triangle, diamond, circle, square, or any suitable shape as desired. Additionally, the reed diffuser can be made like animal, flower, or any natural or artificial shape. Moreover, the color of the reed diffuser can be made in any desirable color such as red, green, yellow, purple, blue, pink, or different combinations of colors.

Such an elegant reed diffuser is inexpensive to be manufactured, easy to store in a sealed container for future use, and flexible to be placed in any location as desired.

In summary, the present invention provides a very simple, straightforward, and portable way to deliver scent through natural airflow by directly applying formulated scents to the screen, without any scent delivery device or to deliver scent through natural or artificial airflow by directly attaching such scent strips to anywhere people want, or using reed diffusers, without any scent delivery device.

Although the screen scent, scent strip and reed diffuser presented here and the methods of using the same according to the present invention has been described in the foregoing specification with considerable details, it is to be understood that modifications may be made to the invention by the inventor, which do not exceed the scope of the appended claims. Modified forms of the present invention done by others skilled in the art to which the invention pertains will be considered infringements of this invention when those modified forms fall within the claimed scope of this invention.

What is claimed is:

1. A novel formulated scent comprises: mineral oil, isopropyl palmitate, dicapryl adipate, fragrance (such as orange-ginger combination), dioctyl sodium sulfosuccinate, butylated hydroxytoluene (BHT), and carrot seed oil.

2. A method to apply said formulated scent of claim 1, wherein directly applied said formulated scent to various screens which will include but not limited to door screens, window screens, tent screens and gazebos exposed to a natural airflow for dispersing such scent over time.

3. There are various ways to directly apply said formulated scents directly to related screens of claim 2, wherein including but not limited to towelettes, fine spray misters, foam applicators.

4. A method to apply said formulated scent of claim 1, wherein a portable scent strip infused or soaked with said formulated scents or natural fragrance is used to deliver scent through natural or artificial airflow by directly attaching said scent strip to various locations as desired.

5. A method to apply said formulated scent of claim 4, wherein said portable scent strip is attached to outside frames of fans/air conditioners within a house or a building.

6. A method to apply said formulated scent of claim 4, wherein said portable scent strip is attached to outdoor structures or furniture including but not limited to gazebos/tents, umbrellas or swing chairs.

7. A method to apply said formulated scent of claim 4, wherein said portable scent strip is made from any suitable material including but not limited to paper, cloth, yarn, plastic, rubber, wood, stone, metal.

8. A method to apply said formulated scent of claim 4, wherein said portable scent strip is made with different sizes, different colors, and different designed shapes as desired or depending on the situation or location.

9. A method to apply said formulated scent of claim 4, wherein said portable scent strip is attached by any conventional means for such attachment including but not limited to using clips, hooks, adhesives and Velcro/elastic ways.

10. A method to apply said formulated scent of claim 1, wherein said portable reed diffuser is placed on suitable outdoor/indoor surfaces such as dining tables/picnic tables etc.

11. A method to apply said formulated scent of claim 1, wherein a reed diffuser infused or soaked with said formulated scent of natural fragrance containing naturally occurring insect repellent is used to deliver scent through natural or artificial airflow by placing reed diffusers in various locations as desired.

12. A method to apply said formulated scent of claim 11, wherein said portable reed diffuser is made from any suitable material including but not limited to paper, cloth, yarn, plastic, rubber, wood, stone, metal.

13. A method to apply said formulated scent of claim 11, wherein said portable reed diffuser is made with different sizes, different colors, and different designed shapes as desired or depending on the situation or location
